(19) United States
(12) Patent Application Publication
(10) Pub. No.: US 2025/0311836 A1
(43) Pub. Date: Oct. 9, 2025

(54) APPLICATOR WITHOUT BRISTLES OR PROJECTIONS

(71) Applicant: GEKA GmbH, Bechhofen (DE)

(72) Inventors: Erika VON JAN, Roth (DE); Margherita DI MAURO, Concorezzo (IT)

(21) Appl. No.: 18/869,760

(22) PCT Filed: Jun. 5, 2023
(86) PCT No.: PCT/EP2023/065010

§ 371 Date: Nov. 26, 2024

(30) Foreign Application Priority Data
Jun. 3, 2022 (DE) 20 2022 103 178.9

(51) Int. Cl. A45D 40/26; A45D 34/04

(52) U.S. Cl. CPC A45D 40/262; A45D 34/042; A45D 2200/10

(57) ABSTRACT

An applicator for applying a pasty or flowable substance is provided. The applicator comprises an applicator element comprising a plurality of rods that are joined at a proximal end of the applicator element. The plurality of rods extend from the proximal end of the applicator element separately to a distal end of the applicator element, where they are reunited. Each of the plurality of rods has a helical shape between the proximal end of the applicator element and the distal end of the applicator element. Each of the plurality of rods has no outwardly protruding bristles or projections.

### CROSS-REFERENCE TO RELATED APPLICATIONS

This application is a U.S. National Stage application of International Application No. PCT/EP2023/065010, filed Jun. 5, 2023, which claims priority to Dutch application Ser. No. 202022103178.9. filed Jun. 3, 2022, the contents of each of which are hereby incorporated by reference.

### BACKGROUND

The disclosure concerns an applicator for applying a pasty or flowable substance using an applicator element, according to the preamble of the main claim.

### Technical Field

Applicators are used for applying makeup or other substances (hereinafter referred to as “pasty or flowable substance”) such as medications to skin, hair. or mucous membranes, for example. inside the nose. They are initially dipped into the substance to be applied. Usually, they capture a portion of the substance on their bristles or their outer circumferential surface. When the applicator is then pulled out of the substance, it must retain the absorbed substance until it is applied to the desired area using rotating and sweeping motions.

### State of the Art

For a conventional applicator to capture and store the substance to be applied, its outer circumferential surface is typically flocked. This gives the applicator a texture in which the substance can be stored in the indentations. depending on its viscosity and surface tension or interfacial tension. However, this flocking process is relatively complex and time-consuming. To flock an applicator. the surface to be flocked must first be coated with an adhesive layer. Then, the flock fibers are applied to the wet adhesive. which must be allowed to cure. Furthermore, flocked applicators can usually only capture substances with relatively high viscosity. In addition the indentations created by the flocking are generally shallow, allowing only a thin film of the substance to be absorbed. Releasing the substance upon deformation of the applicator can also be challenging.

In general, there are currently no suitable applicators that can store a substance and release it upon deformation as intended.

### OBJECTIVE OF THE INVENTION

In light of the above, the objective is to provide an applicator that can store a substance and release it as intended when deformed.

### INVENTIVE SOLUTION

According to the disclosure, this problem is solved with the features of claim 1.

For this purpose, an applicator for applying a pasty or flowable substance is proposed, with an applicator element consisting essentially of a number of rods. These rods are united at a proximal end of the applicator element, preferably fused integrally, and extend separately from each other to a distal end. where they are again united, preferably integrally fused. Each rod has a helical course between the proximal and distal ends. In this context, the “proximal end” refers to the end of the applicator element closest to the stem or stem.

The inventive applicator is characterized by the fact that the mentioned rods do not have any outwardly projecting bristles or protrusions.

The intended substance for the applicator is a flowable cosmetic or pharmaceutical substance, primarily in the form of a serum. It can optionally include eyeliner, concealer, ink, and facial oil. The substance preferably has a low viscosity, e.g., up to 1000 mPas. Ideally, it has a viscosity of 0.2 to 150 mPas. optimally between 0.5 and 75 mPas. In certain other applications, the viscosity ranges from 4000 mPas to 15000mPas. In other applications, the viscosity is between 0.2 and 10000 mPa, optimally 0.5 to 2000 mPa.

The applicator element serves to capture the substance, which can accumulate between the rods and remains there until the user applies the applicator element to the intended surface. There it deforms as intended, breaking the surface tension of the substance collected preferably as a film between the rods. The substance is therefore released in controlled amounts necessary for application. The released substance can then be spread over the application area with the applicator element.

### PREFERRED EMBODIMENTS

A preferred embodiment of the applicator consists of that the rods form an inner basket at the distal end where the substance can be stored, particularly when the applicator element is held vertically downward with its distal end. This ensures secure storage of the substance in the applicator element, which is only released upon deformation.

Additionally, it is particularly preferred if the rods end in a disc at the proximal end that extends radially outward as a drop-catcher, reducing the likelihood of the substance running along a stem that eventually follows. After the release of the substance or after dipping the applicator element into the substance, excess substance not absorbed by the applicator element may drip down and/or run down along the applicator toward the stem. To enable convenient and secure application. it is therefore useful to keep excess substance away from the applicator's grip surface.

It is further preferred that the rods have a generally round cross-section throughout most or all of their length. This offers manufacturing advantages and facilitates better storage of the substance between the rods.

It is also particularly advantageous if adjacent rods at the distal end form a narrowing V-shaped gap just before merging, which gap is area wise so narrow that the substance stored here inside the applicator element cannot overcome the gaps as long as the applicator is not deformed. This results in favorable storage and/or release behavior of the applicator element. The mentioned “proximity area” preferably refers to a distance of at most 20 mm, particularly preferably at most 10 mm. from the point where the rods converge at the distal end.

### ANOTHER INVENTIVE SOLUTION

The disclosure also concerns an applicator according to the preamble of claim 6.

For this, an applicator for applying a pasty or flowable substance is proposed, where the structure forming the applicator element consists of planar, preferably thin-walled and therefore deformable, preferably curved surface sections with openings. These openings form a pouch at the distal end, where the substance can be stored.

The intended substance for the applicator is a flowable cosmetic or pharmaceutical substance, especially in serum form. Eyeliner, concealer, ink, and facial oil can be included as optional ingredients. The substance preferably has a low viscosity, e.g., up to 1000 mPas. Ideally, the viscosity is between 0.2 and 150 mPas, optimally 0.5 to 75 mPas. In certain other applications, the viscosity ranges from 4000 mPas to 15000mPas. In other applications, the viscosity is between 0.2 and 10000 mPa, optimally 0.5 to 2000 mPa.

The applicator element serves to capture the substance, which can thereby accumulate within the applicator element and remains there until the user applies the applicator element to the target surface. There the applicator element deforms as intended in such a way as to break the surface tension of the substance, collected as a film at the openings and between the surface sections. The substance is thus released in appropriate amounts necessary for application and can then be spread over the application area with the applicator element.

### ADDITIONAL PREFERRED EMBODIMENTS

A preferred embodiment of the applicator involves the structure forming the applicator element creating a basket or pouch on the inner side of the distal end of the applicator element in which basket or pouch the substance can be stored, at least when the applicator element is held vertically with the distal end pointing downward. This configuration ensures safe storage of the substance within the applicator element, which will only be released upon deformation of the applicator element

It is also especially preferred if the pouch is movably suspended at the proximal end of the applicator element on leaf-spring-like elastic supports that ideally converge at the proximal end of the applicator element. These elastic supports are preferably created by the arrangement of the mentioned openings in the surface sections and provide the desired deformability of the applicator element.

Additionally, it is especially advantageous if the applicator element has a spoon-like shape, facilitating the application of the substance to certain, especially curved. body areas. such as the cheeks, and allowing easier an areal distribution of the substance.

### ANOTHER INVENTIVE SOLUTION

The disclosure further relates to an applicator according to the preamble of claim 10.

To this end, an applicator for applying a pasty or flowable substance is proposed, wherein the applicator is designed as a flat applicator consisting of more than two structural layers arranged essentially parallel and spaced apart, each structural layer having openings that, under certain conditions, allow the passage of the substance.

The substance intended for this applicator is a flowable cosmetic or pharmaceutical substance, primarily in the form of a serum. Eyeliner, concealer, ink, and face oil may optionally be included. The substance preferably has a low viscosity, e.g., up to 1000 mPas. Ideally, it has a viscosity between 0.2 and 150 mPas, optimally from 0.5 to 75 mPas. In some applications, the viscosity may range between 4000mPas and 15000 mPas, while in other cases, it can range from 0.2 to 10000 mPas, optimally between 0.5 and 2000 mPas.

The applicator element serves to absorb the substance, allowing it to accumulate within the applicator element and remain there until the user applies the applicator element to the surface. There the applicator element deforms as intended, breaking the surface tension of the substance accumulated preferably as a film and/or between the structural lavers. This releases the substance in controlled amounts needed for application. The applicator element can then be used to distribute this released substance over the target surfaceapplicator element.

### ADDITIONAL PREFERRED EMBODIMENTS

A preferred embodiment of the applicator involves spacing adjacent structural layers so closely that they retain the substance between them due to capillary action. This configuration allows a larger quantity of substance to be stored within the applicator element.

Furthermore, it is particularly preferred if the applicator element tapers triangularly from its proximal to its distal end, with multiple structural layers connected at the triangular tip by a common transverse bar. This again facilitates application of the substance to specific body areas and allows more filigree application and distribution when using the triangular tip with the reinforcing transverse bar. Conversely, laying the triangular surface flat against the skin facilitates an areal distribution of the substance.

Moreover, it is especially advantageous if each structural layer consists of outer bordering rods that enclose a free space, which is partially filled by a more filigree rod structure, forming a series of passage openings as breaks in the structural layer which however, are preferably small enough to prevent the substance from passing through, unless the structural layer undergoes external deformation. The series of passage openings therefore realize the already mentioned openings as breaks of the structural layers. These openings as breaks are therefore implicitely formed by the rod structure. In these openings as preaks respectively passage openings the substance can then accumulate, preferably as a film.

### ANOTHER INVENTIVE SOLUTION

The disclosure further pertains to an applicator according to the preamble of claim 14.

For this purpose, an applicator for applying a pasty or flowable substance is proposed, wherein the applicator element consists of a rod bearing several non-circular rings. These rings are attached to the rod in such a way that each ring projects on one side of the rod with a smaller ring segment and on the other side with a larger ring segment.

The substance intended for this applicator is a flowable cosmetic or pharmaceutical substance. with the applicator preferably designed for applying substance to eyebrows, eyelashes, and/or hair. Eyeliner, concealer, ink, and face oil may optionally be included. The substance preferably has a low viscosity, e.g., up to 1000 mPas. Ideally, it has a viscosity between 0.2 and 150 mPas, optimally from 0.5 to 75 mPas. In some applications, the viscosity may range from 4000 mPas to 15000 mPas. while in other cases, it can range from 0.2 to 10000 mPas. optimally between 0.5 and 2000 mPas.

The applicator element is designed to absorb the substance, allowing it to accumulate within the applicator element and remain there until the user applies the applicator element to the target surface. There. the applicator element deforms as intended in such a manner that the surface tension of the substance accumulated in the rings preferably as a film and/or between the rings is broken. This releases the substance in intended amounts needed for application. The released substance can then be distributed over the target surface with the applicator element.

The rings also enable shaping of the eyebrows, eyelashes, and/or hair, as these are carried along especially in the spaces between the rings, when the applicator is correspondingly moved with a stroking and/or twisting motion.

### ADDITIONAL PREFERRED EMBODIMENTS

One preferred embodiment of the applicator is that the orientation of the rings oscillates around a central line, typically extending radially from the rod, preferably up to plus or minus 25°, and more ideally up to plus or minus 15°. This allows for improved application behavior of the substance.

Additionally, it is preferred if the rings oscillate alternately, so that in a pattern repeat. a ring symmetrical to the central line is followed by a ring tilted to one side, followed by a ring tilted to the opposite side, with the pattern continuing in this sequence. A “pattern repeat” here refers to the smallest, self-contained unit which a pattern consists of By repeating and assembling the units. in the case according to the disclosure the sequence of rings with alternating tilt the corresponding pattern is formed.

### ANOTHER INVENTIVE SOLUTION

The disclosure also relates to an applicator according to the preamble of claim 19.

To this end, an applicator for applying a pasty or flowable substance is proposed, wherein the applicator element consists of multiple fields forming a sieve structure that, when the applicator is held in a specific rotated position. collectively form a bowl capable of holding the substance as well under the influence of gravity, wherein the sieve structure has fine clear openings that are small enough to prevent due to the surface tension the substance from passing through completely or essentially by itself.

The substance for which the applicator is intended has the form of a flowable cosmetic or pharmaceutical substance, especially in the form of an oil. Eyeliner, concealer, or ink may optionally be included. The substance preferably has a low viscosity, e.g., in the range of up to 1000 mPas. Ideally, it has a viscosity of 0.2 to 150 mPas, optimally from 0.5 to 75 mPas. In certain other applications, the viscosity is between 4000 mPas and 15000 mPas. In other applications, the viscosity ranges from 0.2 to 10000 mPa. optimally from 0.5 to 2000 mPa.

The applicator element is used to absorb the substance, which can accumulate in the applicator element and remain there until the user applies the applicator element to the surface to be treated. There, the applicator element deforms as intended, breaking the surface tension of the substance accumulated preferably as a film and/or in the shell within the openings. The substance is thus released in the intended amounts needed for application. The released substance can then be distributed on the surface to be treated using the applicator element.

### FURTHER PREFERRED EMBODIMENTS

A particularly preferred embodiment includes at least one additional section on the applicator element that at least partially covers the opening of the bowl.

It is also especially preferred if the additional section comprises at least two main ribs extending along the longitudinal axis, ideally connected in a ladder-like fashion by straight cross ribs. Ideally, the substance can more readily pass through the ribbing of the additional section than through the ribbing of the bowl-shaped sections. By this the bowl as well as the at least one additional section to form various openings where the substance can accumulate or pass through. Due to the easier passage of the substance through the openings of the at least one additional section. the user can on its own dose the released substance based on the orientation or rotation of the applicator.

### ANOTHER INVENTIVE SOLUTION

The disclosure also encompasses an applicator based on the concept of claim 25.

In this design, the applicator for applying a pasty or flowable substance features a smooth-surfaced hollow body with a drop. almond, plum, or zeppelin shape. The body's wall includes multiple, preferably circular or polygonal, holes by which it is sufficiently weakened to deform significantly under forces intentionally encountered during application.

The substance, intended for use with the applicator, takes the form of a fluid cosmetic or pharmaceutical product, particularly in the form of oil. Eyeliner, concealer. ink, and facial oil may be optionally included. The substance preferably has a low viscosity, for example, up to 1000 mPas. Ideally. it ranges between 0.2 to 150 mPas, optimally between 0.5 and 75 mPas. In some application fields, the viscosity ranges from 4000 mPas to 15000 mPas. In others. it ranges from 0.2 to 10000 mPas, with an optimal range between 0.5 and 2000 mPas.

The applicator element is designed to absorb the substance, where it can accumulate until the user applies the applicator to the target surface. There, the applicator intentionally deforms in a way that breaks the surface tension of the substance accumulated preferably as a film in the holes and/or within the hollow body. This releases the substance in specific amounts suitable for application, allowing it to be distributed across the target area with the applicator.

### FURTHER PREFERRED EMBODIMENTS

A preferred embodiment of the applicator features clear diameters of the holes that are so narrow that the mass stored in the inner of the applicator element can exit the applicator only with a not insignificant delay unless the applicator is deformed under application forces. This configuration supports a preferred application behavior.

Moreover, it is especially preferred if the applicator element has a conical section at its distal end free from perforations and/or holes. This conical section may serve as an additional reservoir for the substance.

### BRIEF DESCRIPTION OF THE DRAWINGS

### Example of First Embodiment

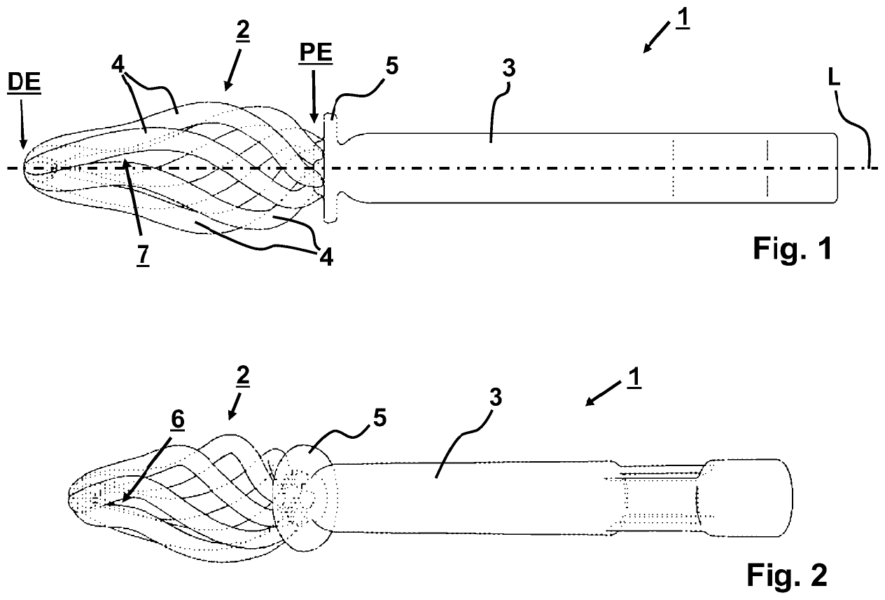

FIG. 1 shows a top view of a first preferred embodiment of the inventive applicator.

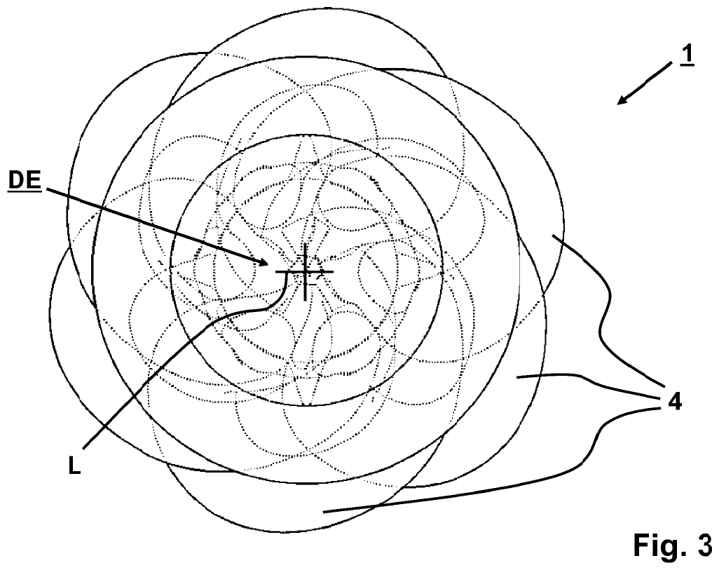

FIG. 2 shows a three-dimensional view of the first preferred embodiment of the

inventive applicator.

FIG. 3 shows a front view of the first preferred embodiment of the inventive applicator.

### Example of Second Embodiment

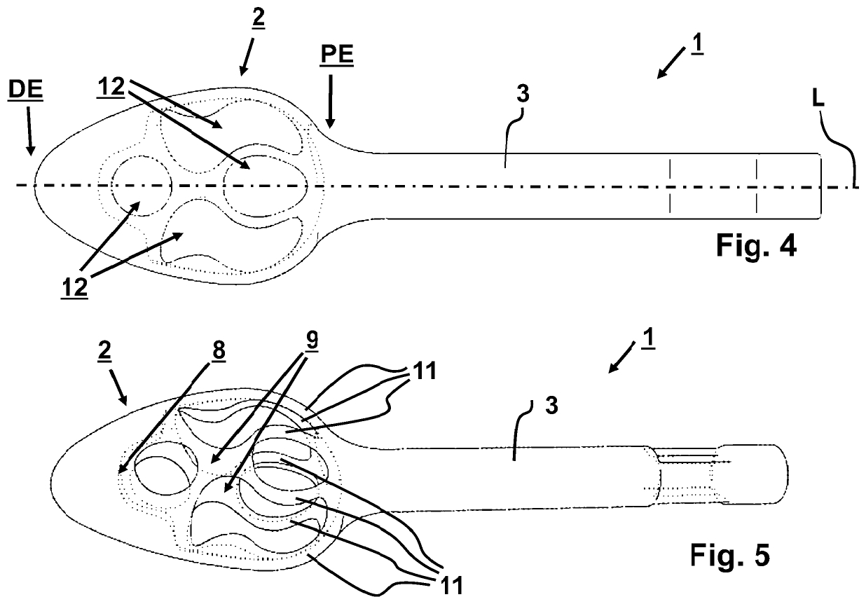

FIG. 4 shows a top view of a second preferred embodiment of the inventive applicator.

FIG. 5 shows a three-dimensional view of the second preferred embodiment of the inventive applicator.

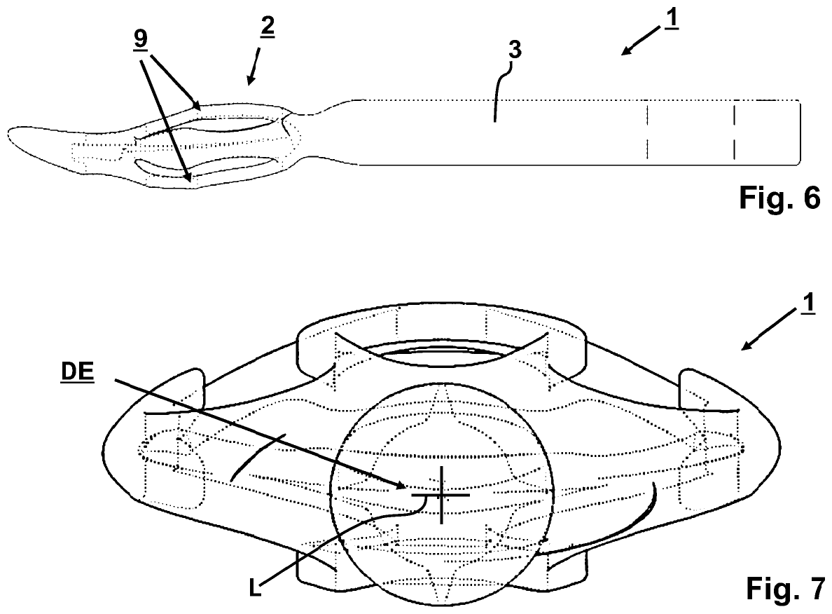

FIG. 6 shows a side view of the second preferred embodiment of the inventive applicator.

FIG. 7 shows a front view of the second preferred embodiment of the inventive applicator.

### Example of Third Embodiment

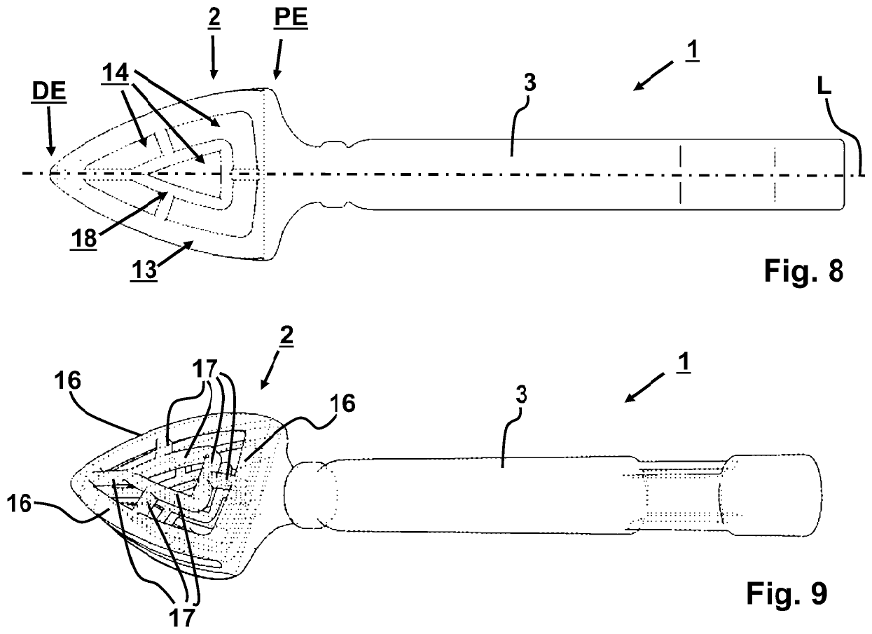

FIG. 8 shows a top view of a third preferred embodiment of the inventive applicator.

FIG. 9 shows a three-dimensional view of the third preferred embodiment of the inventive applicator.

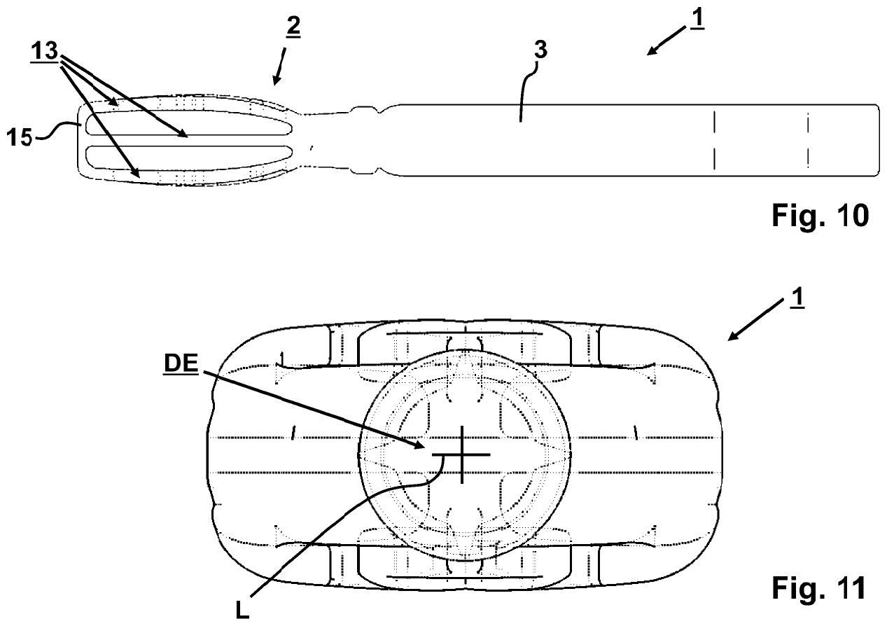

FIG. 10 shows a side view of the third preferred embodiment of the inventive applicator.

FIG. 11 shows a front view of the third preferred embodiment of the inventive applicator.

### Example of Fourth Embodiment

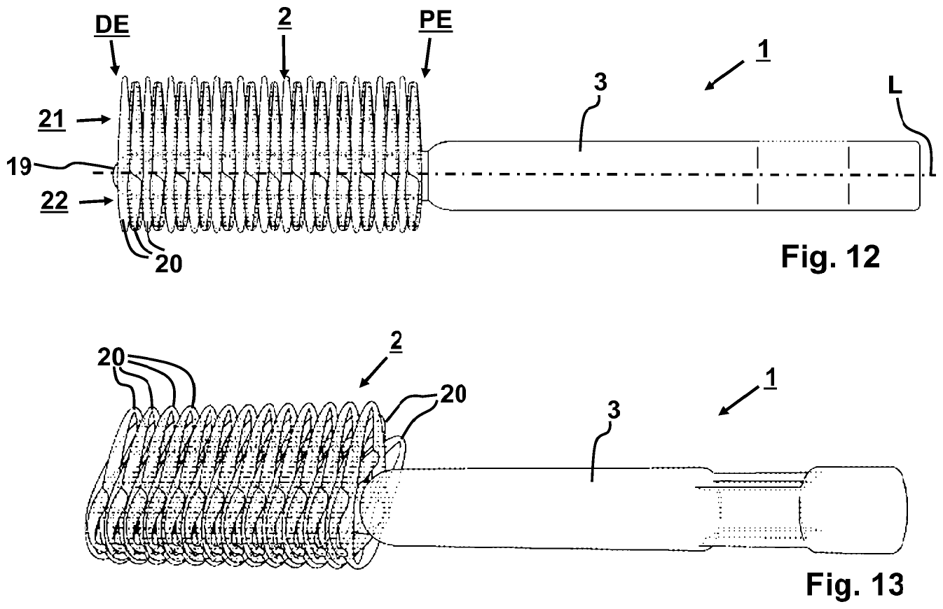

FIG. 12 illustrates a fourth preferred embodiment of an applicator according to the disclosure in a side view.

FIG. 13 shows the fourth preferred embodiment of an applicator in a three-dimensional view.

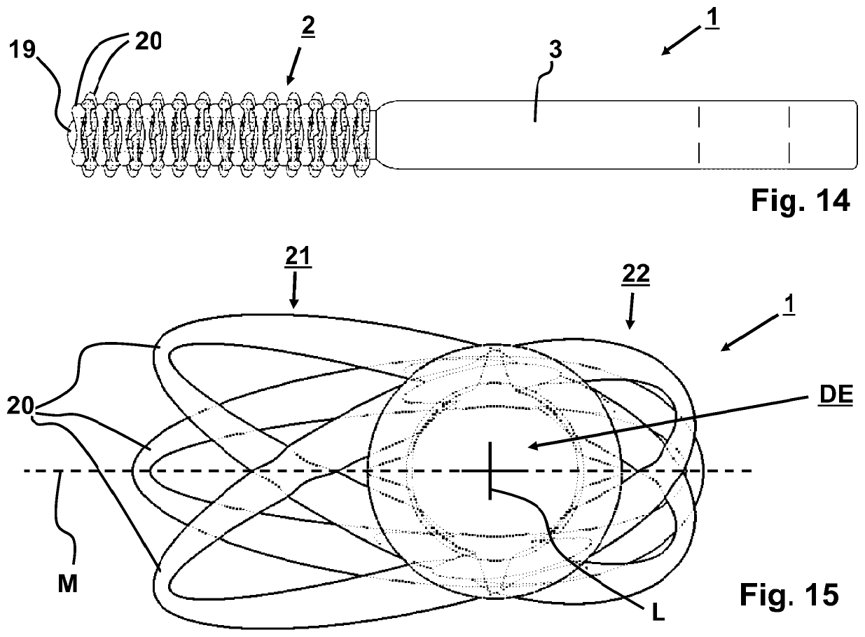

FIG. 14 shows the fourth preferred embodiment of an applicator in a top view.

FIG. 15 shows the fourth preferred embodiment of an applicator in a front view.

### Example of Fifth Embodiment

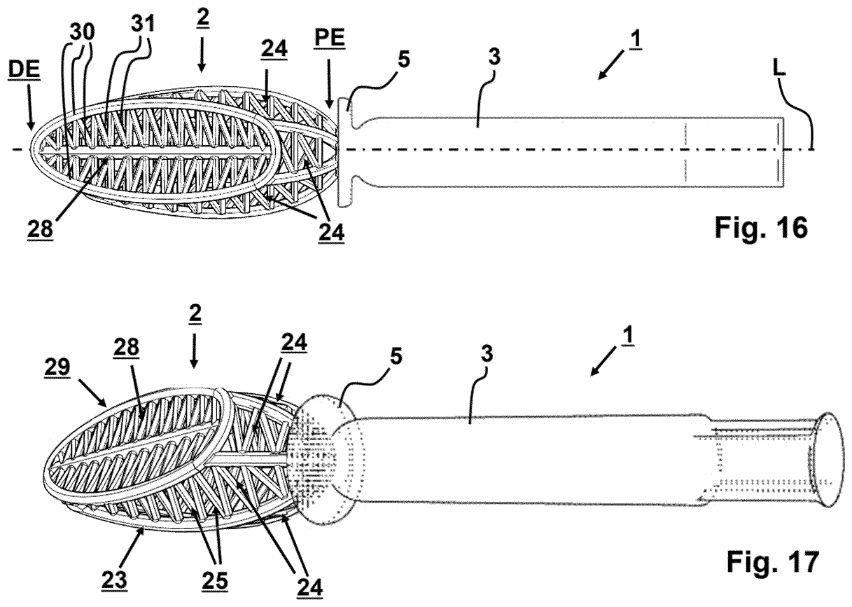

FIG. 16 shows a fifth preferred embodiment of an applicator according to the disclosure in a top view.

FIG. 17 illustrates the fifth preferred embodiment of an applicator in a three-dimensional view.

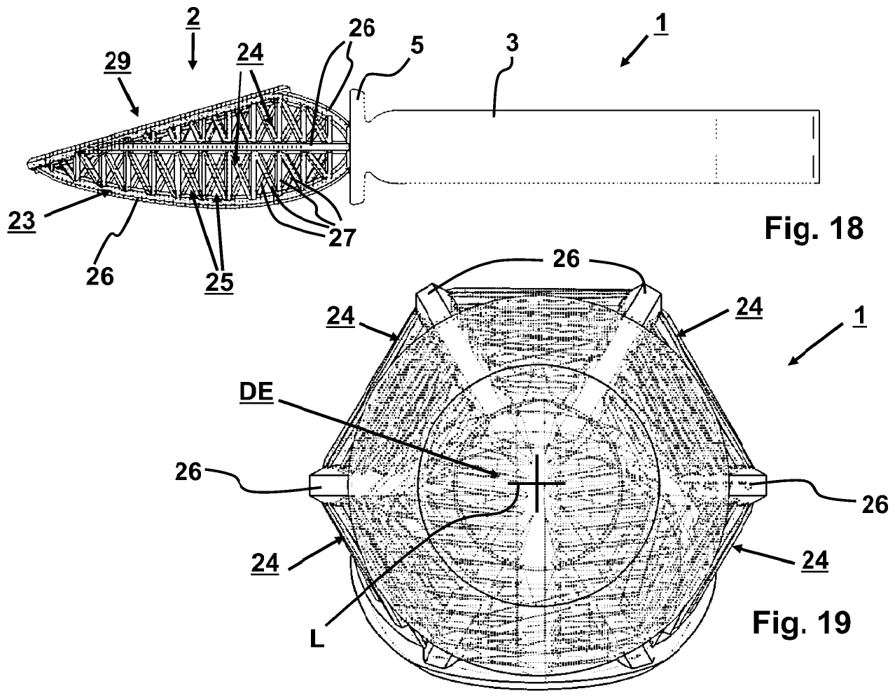

FIG. 18 depicts the fifth preferred embodiment of an applicator in a side view.

FIG. 19 shows the fifth preferred embodiment of an applicator in a front view.

### Example of Sixth Embodiment

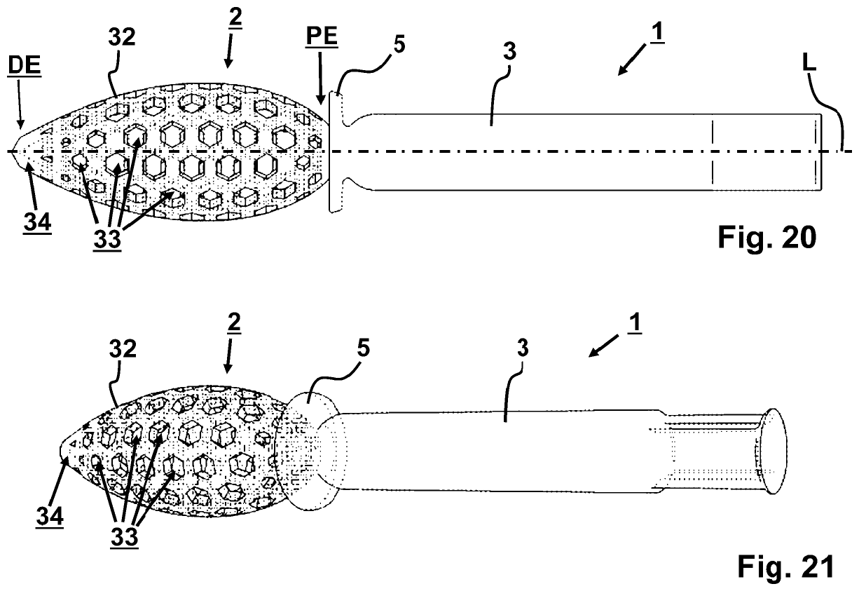

FIG. 20 shows a sixth preferred embodiment of an applicator in a top view.

FIG. 21 illustrates the sixth preferred embodiment of an applicator in a three-dimensional view.

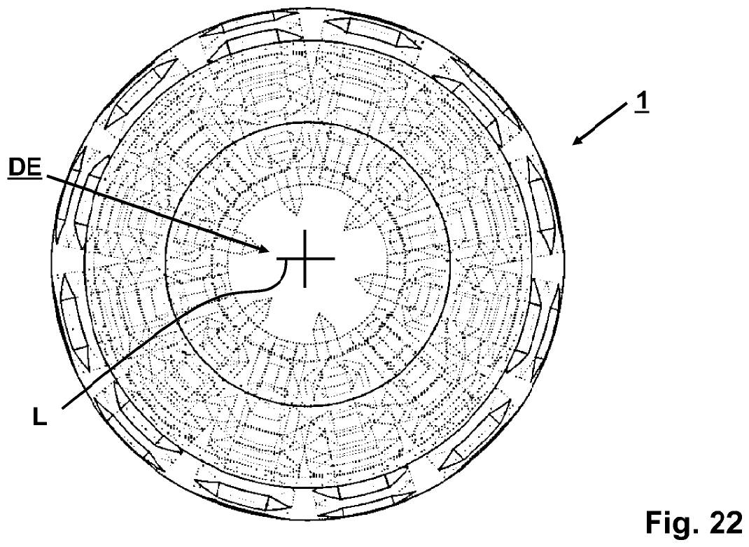

FIG. 22 shows the sixth preferred embodiment of an applicator in a front view.

### PREFERRED EMBODIMENT EXAMPLES

### Example of First Embodiment

FIGS. 1 through 3 show an example of a first embodiment of the applicator 1. with the longitudinal axis L of the applicator marked in FIG. 1. The applicator 1 includes a stem 3 with a disk 5 and an applicator element 2 connected to this disk 5. The applicator element 2 features a proximal end PE (end facing the stem) and a distal end DE (end facing away from the stem).

The applicator element 2 includes a series of helically arranged rods 4 that converge at both the distal end DE and proximal end PE. Near the point where the rods 4 converge at the distal end DE, a V-shaped gap 7 forms between the rods. This arrangement of rods 4 creates a basket structure 6 within the interior of the applicator element 6.

The rods 4 preferably have a circular cross-section. Additionally, the rods 4 are preferably twisted helically such that each rod 4 has a first segment with a curvature forming an inclination angle of 2° to 10° relative to the longitudinal axis L, measured from the distal end DE. This first segment then transitions into a second segment with an opposite curvature, preferably forming an inclination angle of 10° to 30° relative to the longitudinal axis L. In a third segment, the rod 4 transitions with a counter-curvature toward the proximal end PE.

The applicator 1 is generally preferred to be made of plastic. manufactured via additive manufacturing, and designed as a single integral piece.

### Example of Second Embodiment

FIGS. 4 through 7 illustrate an example of a second embodiment of the applicator 1, with the longitudinal axis L of the applicator indicated in FIG. 4. The spoon-shaped applicator 1 includes a stem 3 and an applicator element 2 connected to this stem. The applicator element 2 has a proximal end PE (end facing the stem) and a distal end DE (end facing away from the stem).

The applicator element 2 preferably comprises two curved surface sections 9 that are integrally laying on each other. The surface sections 9 preferably have complementary openings 12. Preferably no openings are present near the distal end DE, where as a consequence a cavity 8 is formed. The surface sections' 9 openings 12 create ridges. which act as leaf-spring-like elastic supports 11 for the cavity.

The applicator 1 is generally preferred to be made of plastic, manufactured via additive manufacturing, and designed as a single integral piece.

### Example of Third Embodiment

FIGS. 8 through 11 show an example of a third embodiment of the applicator 1, with the longitudinal axis L of the applicator indicated in FIG. 8. The flat-shaped applicator 1 includes a stem 3 and an applicator element 2 connected to this stem. The applicator element 2 has a proximal end PE (end facing the stem) and a distal end DE (end facing away from the stem) and is constructed with a triangular cross-sectional shape.

The applicator element 2 includes multiple structural layers 13 arranged one above the other. These structural layers comprise bordering rods 16 arranged in a triangular formation. Within these bordering rods 16, filigree rods 17 form a rod structure 18. with several openings 14 functioning as passage openings. At the triangular tip of the applicator element 2, a crossbar 15 connects the multiple structural layers 13.

The rod structure 18 is preferably designed so that within the bordering rods 16, a triangular form composed of filigree rods 17 is created and connected to the bordering rods 16 by additional filigree rods 17.

The applicator 1 is generally preferred to be made of plastic. manufactured via additive manufacturing. and designed as a single integral piece.

### Example of Fourth Embodiment

FIGS. 12 through 15 illustrate an example of a fourth embodiment of the applicator 1, with the longitudinal axis L of the applicator indicated in FIG. 12. The applicator 1 comprises a stem stem3 and an applicator element 2 connected to this stem. The applicator element 2 has a proximal end PE (the end facing the stem) and a distal end DE (the end opposite the stem).

The applicator element 2 includes a preferably central rod 19, which is preferably arranged coaxially to the stem 3 and directly connects to it. The rod 19 thereby supports several closed rings 20. arranged along the longitudinal axis of the rod 19 and embracing it. The rings 20 are preferably positioned on the rod 19 in such a manner that each ring stands vertically along the longitudinal axis L with a larger ring section 21 and a smaller ring section 22.

It is also preferred that the rings 20 alternate around a radial central line M from the rod 19, preferably by up to plus or minus 25° and better by plus or minus 15°, as vividly shown in FIG. 15. Often, it is preferred for one ring 20 with a positive angle relative to the central line M to be followed by a ring 20 with a negative angle, then by a ring 20 with no angle. continuing this sequence essentially along the entire rod 19.

The applicator I is generally preferred to be made of plastic, manufactured via additive production, and integrally constructed as a single piece.

### Example of Fifth Embodiment

FIGS. 16 through 19 show an example of a fifth embodiment of the applicator 1, with the longitudinal axis L of the applicator depicted in FIG. 16. The applicator 1 includes a stem 3 with a disc 5 and an applicator element 2 attached to this disc. The applicator element 2 has a proximal end PE (the end facing the stem) and a distal end DE (the end opposite the stem).

The applicator element 2 comprises multiple segments 24 arranged angularly to form a shell 23. These segments 23 preferably consist of two main ribs 26, extending primarily along the longitudinal axis L. connected by cross ribs 27. which preferably follow a zig-zag path. This arrangement creates the mentioned sieve structure with multiple fine clear openings 25. The resulting shell 23 preferably takes on a boat shape, with a more pointed bow and a blunter stern.

This conceptual shell 23 has a shell opening 29, ideally partially closed by at least one additional field 28. This additional field 28 consists. in turn, of main ribs 30, connected by cross ribs 31, which are arranged straight rather than in a zig-zag pattern. Thus, a sieve structure with openings is also created in this additional field 28.

The applicator 1 is generally preferred to be made of plastic, manufactured via additive production, and integrally constructed as a single piece.

### Example of Sixth Embodiment

FIGS. 20 through 21 illustrate an example of a sixth embodiment of the applicator 1, with the longitudinal axis L of the applicator depicted in FIG. 20. The applicator 1 includes a stem 3 with a disc 5 and an applicator element 2 attached to this disc. The applicator element 2 has a proximal end PE (the end facing the stem) and a distal end DE (the end opposite the stem).

The applicator element 2 comprises a wall 32 that forms a teardrop shape, resulting in a smooth-surfaced hollow body. The wall 32 is perforated by multiple holes 33 along its entire surface. The tip of the applicator element 2 at its distal end DE preferably has a conical area without holes.

The applicator 1 is generally preferred to be made of plastic, manufactured via additive production, and integrally constructed as a single piece.

1. An applicator for applying a pasty or flowable substance, the applicator comprising:
an applicator element comprising a plurality of rods that are joined at a proximal end (PE) of the applicator element, the plurality of rods extending from there-the proximal end of the applicator element separately to a distal end of the applicator element, where they are reunited,
each of the plurality of rods having a helical shape between the proximal end of the applicator element and the distal end of the applicator element, characterized and
each of the plurality of rods having no outwardly protruding bristles or projections.

2. The applicator according to claim 1, wherein the plurality of rods form a basket on an inner side at the distal end of the applicator element, in which substance can be stored when the applicator element is held with the distal end of the applicator element facing vertically downwards.

3. The applicator according to claim 1, wherein the plurality of rods end in a disk at the proximal end of the applicator element, which extends radially outward as a drop catcher that reduces the tendency for substance to run along an adjacent stem.

4. The applicator according to claim 1, wherein the each of plurality of rods has a substantially round cross-section over its entire length or over the a majority of their its length.

5. The applicator according to claim 1, wherein immediately adjacent rods of the plurality of rods form, at the distal end of the applicator element, a narrowing V-shaped gap before they unite, and the gap narrows to an extent that the substance stored on an inside of the applicator element cannot pass through the gap unless the applicator element is deformed.

6. The applicator according to claim 1, wherein a structure forming the applicator element consists of flat and deformable, surface sections with openings that form a pouch at the distal end of the applicator element, in which substance can be stored.

7. The applicator according to claim 6, wherein the structure forming the applicator element forms a basket or a pouch on an inside of the applicator element at the distal end of the applicator element, in which substance can be stored, at least when the applicator element is held with the distal end of the applicator element facing vertically downwards.

8. The applicator according to claim 7, wherein the pouch is movably suspended at the proximal end of the applicator element on leaf-spring-like elastic supports that unite at the proximal end of the applicator element (2).

9. The applicator according to claim 6, wherein the applicator element has a spoon-like design.

10. The applicator according to claim 1, wherein the applicator is a flat applicator consisting of more than two structural layers arranged substantially parallel and spaced apart from each other, each of the structural layers having openings that allow passage of substance.

11. The applicator according to claim 10, wherein immediately adjacent layers of the more than two structural layers are spaced so closely that they hold substance therebetween due to capillary effect.

12. The applicator according to claim 10, wherein the applicator element tapers in a triangular shape from the proximal end of the applicator element to the distal end of the applicator element, with the more than two structural layers being connected by a common crossbar at a tip of the triangular shape.

13. The applicator according to claim 10, wherein each layer of the more than two structural layers consists of rods that border the outside of the layer, forming a free space therebetween, which is partially filled by a rod structure, such that the layer has a number of openings in the form of through holes that are small enough to prevent the substance from passing through them unless the layer undergoes external deformation.

14. The applicator according to claim 1, wherein the applicator element comprises a first rod that carries a number of non-circular rings, the rings being attached to the first rod in such a way that each ring extends on one side of the first rod with a first ring section and on an opposite side of the rod with a second ring section that is larger than the first ring section.

15. The applicator according to claim 14, wherein each of the rings has an elliptical, oval, or plum shape.

16. The applicator according to claim 14, wherein an orientation of the rings oscillates around a central line extending radially from the first rod.

17. The applicator according to claim 16, wherein the rings oscillate alternately, such that for each ring symmetrically placed on the central line, a first ring follows that is tilted to one side of the central line, followed by a second ring tilted to the other side, and then a third ring symmetrically placed.

18. The applicator according to claim 14, wherein for each of the rings, a cross-sectional diameter of a structure forming the ring decreases as the ring moves away from the first rod and reaches a minimum at an apex of the ring.

19. The applicator according to claim 1, wherein the applicator element comprises a plurality of fields forming a sieve structure, the plurality of fields together forming a shell when the applicator is held in a corresponding twisted position, and which can hold substance under the influence of gravity, the sieve structure having fine openings so that the substance cannot pass through the fine openings due to surface tension.

20. The applicator according to claim 19, wherein each field of the plurality of fields comprises two main ribs extending substantially along a longitudinal axis with two adjacent fields of the plurality of fields sharing a common main rib.

21. The applicator according to claim 19, wherein the shell comprises at least three fields arranged at angles to each other.

22. The applicator according to claim 19, wherein the shell has a boat-like shape.

23. The applicator according to claim 19, wherein the applicator element has at least one additional field that partially closes an opening of the shell.

24. The applicator according to claim 23, wherein the at least one additional field comprises at least two main ribs extending along a longitudinal axis connected in a ladder-like pattern by straight transverse ribs and allowing substance to pass through ribbing of the additional field more easily than through ribbing of the plurality of fields forming the shell.

25. The applicator according to claim 1, wherein the applicator element is an externally smooth-walled hollow body with a drop-shaped, almond-shaped, plum-shaped, or airship-shaped design, and a wall of the applicator element having a plurality of circular or polygonal holes and being weakened such that under forces occurring during application, the wall can be deformed.

26. The applicator according to claim 25, wherein a diameter of the holes is such that the mass stored inside the applicator body can exit the applicator only with a time delay, unless the applicator is deformed under forces that occur during application.

27. The applicator according to claim 25, wherein the applicator body has a conical section at the distal end of the applicator element, the conical section being free of openings or holes.
